(19) United States
(12) Patent Application Publication
(10) Pub. No.: US 2006/0111611 A1
(43) Pub. Date: May 25, 2006

(54) Endoscope sleeve dispenser

(75) Inventors: Amram Eizenfeld, Menashe (IL); Golan Salman, Tirat Hacarmel (IL)

Correspondence Address: BRUCE E. LILLING;LILLING & LILLING P.C., P.O. BOX 560, GOLDEN BRIDGE, NY, 10526, US

(73) Assignee: Sightline Technologies Ltd., Haifa (IL)

(21) Appl. No.: 10/524,867

(22) PCT Filed: Aug. 7, 2003
(86) PCT No.: PCT/IL03/00661

§ 371 Date: Feb. 14, 2005

Related U.S. Application Data
(60) Provisional application No. 60/404,110, filed on Aug. 15, 2002.

(51) Int. Cl. A61B 1/00

(52) U.S. Cl. 600/124

(57) ABSTRACT

Apparatus (20) for sheathing an endoscope (22) includes a dispenser (24), having entry and exit ports (36, 39) defining a transit passage through which the endoscope may pass, and a flexible sleeve (26), at least a portion of which is bunched in a vicinity of the dispenser. The sleeve includes a distal end (28), which is closed, and a proximal end, which is open and fixed to the dispenser so that as the endoscope is advanced in a distal direction through the transit passage, the endoscope enters into the sleeve through the proximal end and engages the distal end of the sleeve, thus causing the bunched portion of the sleeve to be extended so as to cover a distal part of the endoscope that protrudes through the exit port.

### CROSS-REFERENCE TO RELATED APPLICATION

This application claims the benefit of U.S. Provisional Patent Application 60/404,110, filed Aug. 15, 2002, which is incorporated herein by reference.

### FIELD OF THE INVETION

The present invention relates generally to endoscopy, and specifically to the use of a disposable sleeve to cover an endoscope during insertion of the endoscope into a body passage.

### BACKGROUND OF THE INVENTION

The use of a disposable sleeve (also referred to as a sheath) to cover an endoscope is well known in the art. Flexible endoscopes, such as colonoscopes, are notoriously difficult to clean and disinfect properly after use, leading to problems of cross-contamination between patients. These problems can be avoided by covering the endoscope with a single-use sleeve, which is discarded after use.

For example, U.S. Pat. No. 4,646,722, whose disclosure is incorporated herein by reference, describes a protective endoscope sheath and a method for installing such sheaths. The sheath has the form of a flexible tube, which fits tightly over the elongated core (i.e., the insertion tube) of the endoscope. The flexible tube has a transparent window near its distal end, positioned in front of the viewing window of the endoscope. Channels for taking biopsies, injecting air or injecting water may extend along the endoscope, either inside or outside the sheath. The patent describes a number of methods for installing the sheath on the endoscope before inserting the endoscope into the body. One of the methods involves inflating the sheath with pressurized gas, causing the sheath to expand, so that the sheath slips easily onto the endoscope core. The source of pressurized gas is then removed, and the sheath collapses to a tight fit on the endoscope.

U.S. Pat. No. 6,485,409, whose disclosure is incorporated herein by reference, describes a method for advancing an endoscopic probe through the lower gastrointestinal tract of a patient by inflation of a flexible sleeve coupled to the probe. One end of the sleeve is anchored, typically at or adjacent to the patient's anus. As the sleeve is inflated, preferably using a pressurized gas, the probe is propelled forward, and the sleeve is fed out gradually between the probe and the anus. The portion of the sleeve that is inflated expands radially outward and remains substantially stationary relative to the intestinal wall as long as it is inflated. Longitudinal motion of the sleeve relative to the wall generally occurs only at and adjacent to the probe itself. The probe is thus advanced easily, and trauma to the gastrointestinal tract is minimized. To remove the probe, the sleeve is deflated and is used to pull the probe back out through the anus.

### SUMMARY OF THE INVENTION

Embodiments of the present invention provide improved methods and devices for fitting a disposable sleeve over an endoscope. In these embodiments, the sleeve is deployed over the endoscope as the endoscope is inserted into a body passage, rather than in a separate, preliminary operation as in sheathing systems known in the art. The sleeve is initially attached, in a bunched, compact form, to a dispenser, which is typically disposable, as well. The distal end of the endoscope is inserted through an entry port in the dispenser into the bunched sleeve, and engages the distal end of the sleeve. The dispenser is placed next to a body opening, and the distal end of the endoscope, covered by the sleeve, is then advanced through an exit port of the dispenser into the body passage. Advancing the endoscope in this manner causes the sleeve to feed out gradually so that it covers the part of the endoscope that is inside the body passage, thus protecting the endoscope from contamination. The sleeve may be inflated in order to reduce friction between the sleeve and the endoscope as the endoscope advances.

When the endoscope is retracted from the body passage, a capture mechanism in the dispenser catches the proximal end of the sleeve so that the sleeve is stripped off the endoscope and gathered within the dispenser. Thus, the contaminated sleeve remains in the dispenser, so that areas outside the body, including the operator's hands, the endoscope controls and the examining table, never come into contact with the contamination. Alternatively or additionally, the dispenser may contain an outer sleeve, which is extended over the contaminated endoscope sleeve as the endoscope is retracted from the body. The sleeve may be deflated while the endoscope is retracted in order to ensure that the sleeve slides back out of the body passage along with the endoscope and is captured evenly by the dispenser.

Embodiments of the present invention are particularly useful in colonoscopy, but the principles of the present invention may similar be applied in other areas of endoscopic examination, surgery and other invasive medical procedures.

There is therefore provided, in accordance with an embodiment of the present invention, apparatus for sheathing an endoscope, including:

a dispenser, having entry and exit ports defining a transit passage through which the endoscope may pass; and

a flexible sleeve, at least a portion of which is bunched in a vicinity of the dispenser, the sleeve including a distal end, which is closed, and a proximal end, which is open and fixed to the dispenser so that as the endoscope is advanced in a distal direction through the transit passage, the endoscope enters into the sleeve through the proximal end and engages the distal end of the sleeve, thus causing the bunched portion of the sleeve to be extended so as to cover a distal part of the endoscope that protrudes through the exit port.

Typically, the dispenser is adapted to be placed so that the exit port is adjacent to a body opening of a patient, whereby the distal part of the endoscope that is covered by the sleeve extends through the body opening into a body passage of the patient.

In a disclosed embodiment, the bunched portion of the sleeve is adjacent to the distal end of the sleeve, and the sleeve is adapted to extend away from the bunched portion in a proximal direction so as to cover the distal part of the endoscope as the endoscope is advanced. In an aspect of the invention, the dispenser is adapted to capture the sleeve as the endoscope is retracted through the transit passage in the proximal direction, so that the sleeve is removed from a proximal part of the endoscope that has been retracted through the entry port, and the removed sleeve is gathered in the dispenser. The sleeve may be adapted so that after the endoscope has been retracted, whereby the sleeve is removed from the proximal part of the endoscope, a further part of the sleeve extends away from the bunched portion in the proximal direction so as to cover the distal part of the endoscope as the endoscope is again advanced through the transit passage.

In some embodiments, the dispenser is adapted to capture the sleeve as the endoscope is retracted through the transit passage in a proximal direction, so that the sleeve is removed from a proximal part of the endoscope that has been retracted through the entry port, and the removed sleeve is gathered in the dispenser. Typically, the dispenser is adapted to gather substantially all of the sleeve, so that after the distal part of the endoscope has been retracted through the entry port, substantially all of the sleeve is contained within the dispenser.

In another embodiment, the apparatus includes an external sleeve, fixed to the dispenser, which is adapted to be extended from the dispenser when the endoscope is retracted through the transit passage, so that the external sleeve covers the flexible sleeve that was extended to cover the distal part of the endoscope. Typically, the dispenser includes a proximal section, which defines the entry port and to which the flexible sleeve is fixed, and a distal section, which defines the exit port and to which the external sleeve is fixed, and wherein the distal section is adapted to be moved away from the proximal section of the dispenser so as to extend the external sleeve over the flexible sleeve when the endoscope is retracted through the transit passage.

In an aspect of the invention, the sleeve is adapted to be inflated while the endoscope is advanced through the transit passage, and to be deflated while the endoscope is retracted proximally through the transit passage. In one embodiment, the dispenser includes a channel, communicating with the sleeve, for inflating the sleeve while the endoscope is advanced and for applying suction to the sleeve while the endoscope is retracted. Typically, the entry port is adapted to fit snugly around the endoscope so as to prevent escape of pressure through the entry port when the sleeve is inflated.

In some embodiments, the apparatus includes a working channel extending through the sleeve alongside the endoscope, the working channel including distal and proximal extremities, wherein the distal extremity is fixed to the distal end of the sleeve, and the proximal extremity protrudes from the dispenser. Typically, the apparatus includes a sealing element, which is adapted to seal the proximal extremity of the working channel while the endoscope is removed from the dispenser.

Alternatively or additionally, when the endoscope includes a working channel having distal and proximal outlets, and the apparatus may include an internal sleeve, which is adapted to be inserted through the working channel, the internal sleeve including distal and proximal extremities, wherein the distal extremity is fixed to the distal end of the sleeve, and the proximal extremity protrudes from the proximal outlet of the working channel.

There is also provided, in accordance with an embodiment of the present invention, apparatus for endoscopy, including:
an endoscope, which is adapted to be inserted into a body passage of a patient; and
a sleeve assembly, which includes:
   a dispenser, having entry and exit ports defining a transit passage through which the endoscope may pass; and
   a flexible sleeve, at least a portion of which is bunched in a vicinity of the dispenser, the sleeve including a distal end, which is closed, and a proximal end, which is open and fixed to the dispenser so that as the endoscope is advanced in a distal direction through the transit passage, the endoscope enters into the sleeve through the proximal end and engages the distal end of the sleeve, thus causing the bunched portion of the sleeve to be extended so as to cover a distal part of the endoscope that protrudes through the exit port.

In some embodiments, the apparatus includes a channel, communicating with the sleeve, for inflating the sleeve while the endoscope is advanced in the distal direction through the transit passage and for applying suction to the sleeve while the endoscope is retracted proximally through the transit passage. The channel may be contained within the endoscope or, alternatively, within the dispenser, alongside the endoscope.

In a disclosed embodiment, the sleeve assembly includes a working channel extending through the sleeve alongside the endoscope, the working channel including distal and proximal extremities, wherein the distal extremity is fixed to the distal end of the sleeve, and the proximal extremity protrudes from the dispenser. In another embodiment, the endoscope includes a working channel having distal and proximal outlets, and the sleeve assembly includes an internal sleeve, which is adapted to be inserted through the working channel, the internal sleeve including distal and proximal extremities, wherein the distal extremity is fixed to the distal end of the sleeve, and the proximal extremity protrudes from the proximal outlet of the working channel.

There is additionally provided, in accordance with an embodiment of the present invention, a method for protecting an endoscope from contamination, including:

providing a flexible sleeve including a distal end, which is closed, and a proximal end, which is open, wherein at least a portion of the sleeve is bunched in a compaction region;

inserting a distal part of the endoscope into the proximal end and through the bunched portion of the sleeve so as to engage the distal end; and

advancing the endoscope through a body opening of a patient into a body passage while extending the bunched portion of the sleeve so as to cover the distal part of the endoscope that extends through the body opening into the body passage.

The present invention will be more fully understood from the following detailed description of the embodiments thereof, taken together with the drawings in which:

### BRIEF DESCRIPTION OF THE DRAWINGS

FIG. 1 is a schematic, partly sectional illustration of a sleeve assembly for an endoscope, in accordance with an embodiment of the present invention;

FIG. 2 is a schematic, pictorial illustration, partly sectional and partly cutaway, showing insertion of an endoscope into the sleeve assembly of FIG. 1, in accordance with an embodiment of the present invention;

FIGS. 3-6 are schematic, partly sectional illustrations showing different stages in the insertion of an endoscope into a sleeve and withdrawal of the endoscope from the sleeve, in accordance with an embodiment of the present invention;

FIG. 7 is a schematic, partly sectional illustration of an endoscope with a working channel in a sleeve assembly, in accordance with an embodiment of the present invention;

FIG. 8 is a schematic, partly sectional illustration of an endoscope in a sleeve assembly with a working channel, in accordance with another embodiment of the present invention;

FIG. 9 is a schematic, partly sectional illustration of a sleeve assembly for an endoscope, in accordance with a further embodiment of the present invention;

FIG. 10 is a schematic, partly sectional illustration showing the insertion of an endoscope into a sleeve provided by the sleeve assembly of FIG. 9, in accordance with an embodiment of the present invention; and

FIG. 11 is a schematic, partly sectional illustration showing a stage in the removal of an endoscope from the sleeve assembly of FIG. 9, in accordance with an embodiment of the present invention.

### DETAILED DESCRIPTION OF EMBODIMENTS

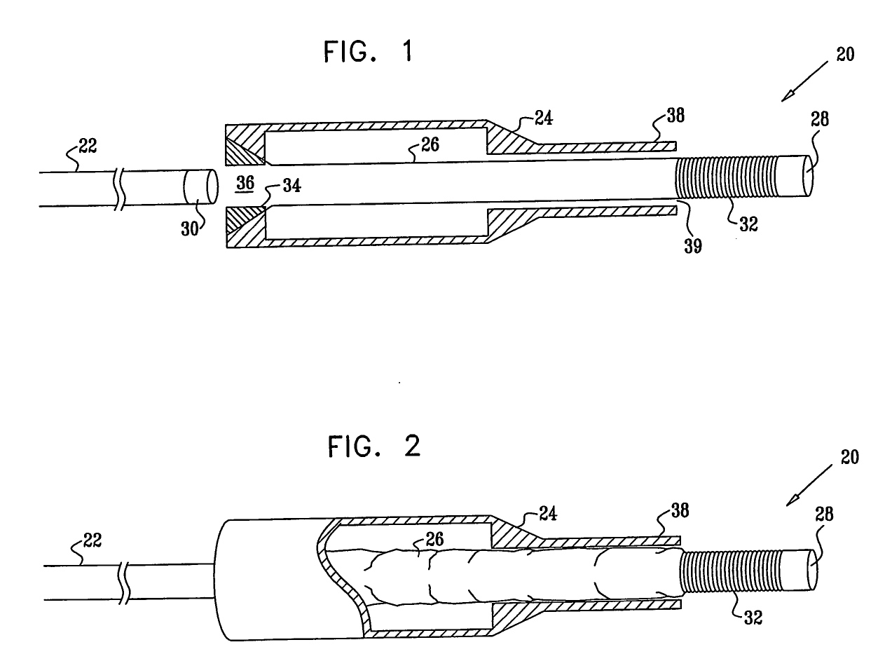

FIG. 1 is a schematic, partly sectional illustration of a sleeve assembly 20 for an endoscope 22, in accordance with an embodiment of the present invention. Assembly 20 comprises a dispenser 24 and a flexible sleeve 26. Typically, dispenser 24 comprises a rigid plastic material, such as PVC, while sleeve 26 comprises a flexible, biocompatible plastic, such as polyamide, having a thickness of about 20 μm. In an exemplary embodiment, endoscope 22 is a colonoscope having an outer diameter of about 13 mm, while sleeve 26 has a diameter of about 20 mm.

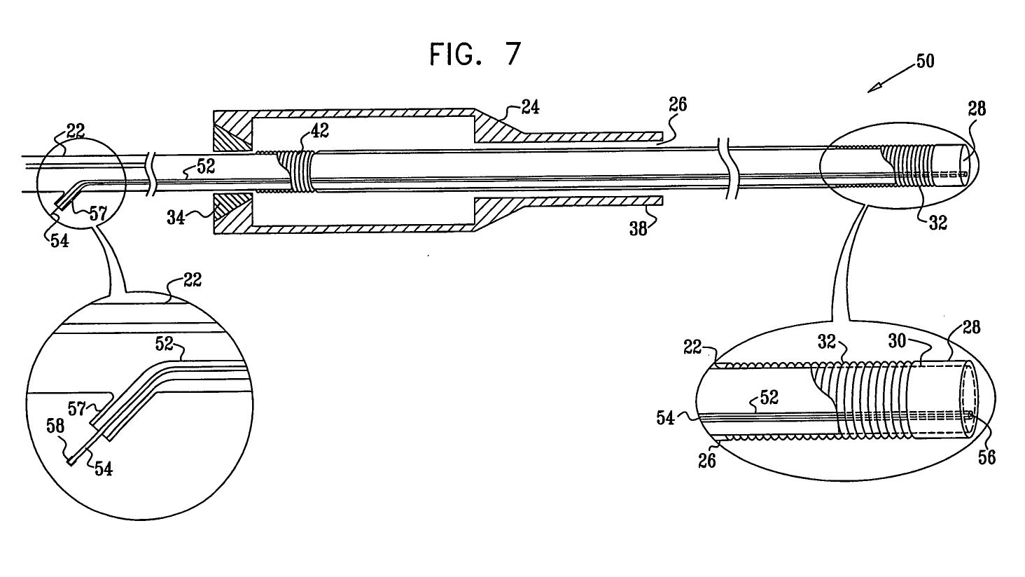

Endoscope 22 has a distal end 30, which typically includes illumination and viewing optics (not shown) and, optionally, distal openings of one or more working channels (as shown, for example, in FIG. 7). When the endoscope is inserted into sleeve 26, the distal end of the endoscope engages a distal end 28 of the sleeve, which comprises a transparent window through which the viewing optics of the endoscope may observe the interior of a body passage, such as the colon. The length of sleeve 26 is typically equal at least to the length of the endoscope, and may be greater. Initially, however, most of the length of the sleeve is bunched in a distal compaction section 32 located in a vicinity of dispenser 24, either inside the dispenser or outside, as shown in FIG. 1. Typically, the bunched sleeve in section 32 is folded upon itself in an accordion fold, like the bendable portion of a plastic drinking straw. The proximal end of the sleeve is held in dispenser 24 by an anchor 34, which is press-fit or glued into the base of the dispenser. The anchor contains an entrance port 36 through which the endoscope is inserted into the dispenser, while a neck 38 of the dispenser contains an exit port 39. The entrance and exit ports thus define a transit passage for the endoscope through the dispenser.

FIG. 2 is a schematic, partly cutaway illustration showing the initial stage of insertion of endoscope 22 into sleeve 26, in accordance with an embodiment of the present invention. The endoscope is inserted into assembly 20 through entrance port 36 (FIG. 1) and is advanced through the sleeve until distal end 30 of the endoscope engages distal end 28 of the sleeve. Anchor 34 fits snugly around the endoscope, for reasons described hereinbelow, and may therefore be lubricated to permit the endoscope to move smoothly in and out of dispenser 24. Sleeve 26, however, fits loosely around the endoscope, as illustrated in this figure, so that the initial insertion is accomplished without difficulty and without requiring special means for fitting the sleeve onto the endoscope. The endoscope and sleeve are now ready for use in an endoscopic examination. At this stage, the endoscope operator (typically a physician) places dispenser 24 adjacent to a body opening, such as the anus, through which the endoscope is to be inserted into the body passage, and inserts the distal end of the endoscope, covered by sleeve 26, through the opening.

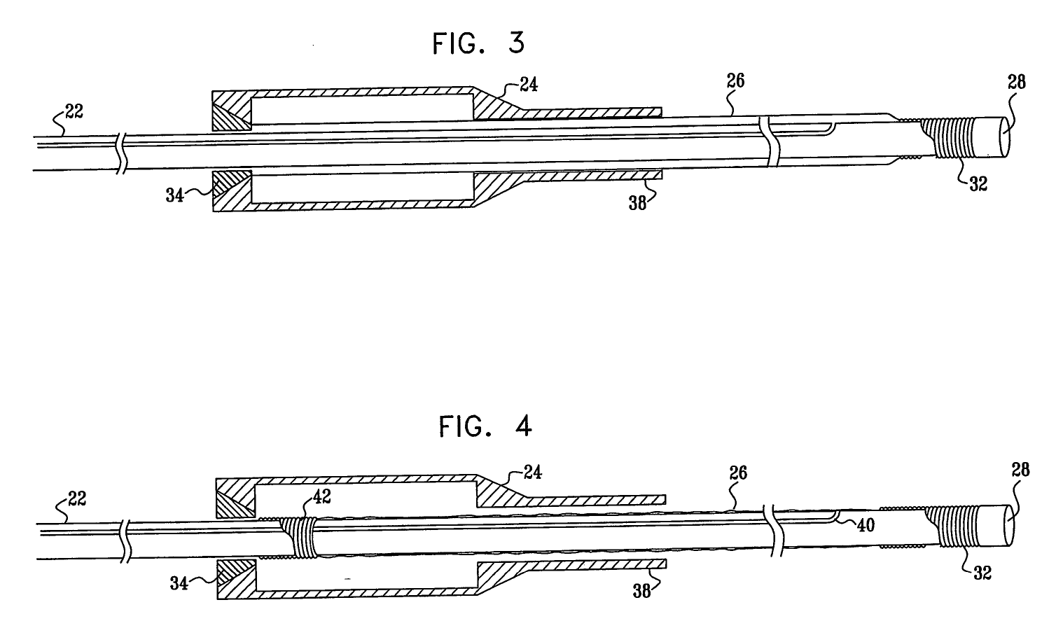

FIG. 3 is a schematic, partly sectional illustration showing endoscope 22 in sleeve 26 as the endoscope within the sleeve is advanced into the body passage, in accordance with an embodiment of the present invention. In order to advance the endoscope, sleeve 26 is inflated with pressurized gas, such as air or carbon dioxide, to a pressure of about 0.4 Bar. Inflation of the sleeve may be accomplished through a channel 40 in endoscope 22 that is provided for this purpose. Alternatively, the sleeve may be inflated through a separate channel (not shown) in dispenser 24, such as a channel passing through anchor 34 adjacent to entrance port 36. Because distal end 28 of sleeve 26 is sealed shut, while anchor 34 fits snugly around the endoscope at the proximal end of the sleeve, the pressurized gas remains in the space between the sleeve and the endoscope and does not leak out in any substantial quantity. The gas pressure (and suction, as described below) may be applied automatically, in response to motion of endoscope 22. Alternatively or additionally, the pressure and suction may be controlled manually by the operator of the endoscope.

This inflation of sleeve 26 pushes the sleeve away from the endoscope surface, as shown in FIG. 3, so that the endoscope can be advanced through the sleeve with minimal friction. As the endoscope advances into the body passage, sleeve 26 unfolds from compaction section 32 near the distal end of the sleeve, and thus extends proximally along the endoscope from section 32. It will be observed that the unfolded portion of the sleeve, which is anchored by dispenser 24, remains stationary relative to the wall of the body passage through which the endoscope is advanced. The only substantial relative motion of the sleeve relative to the wall of the body passage occurs in the area of distal end 28. Consequently, trauma and discomfort to the patient due to friction between the endoscope surface (or the endoscope sheath) and the body passage are reduced relative to methods of endoscopy known in the art. Of course, there is no contact at all between the advancing endoscope itself and the wall of the body passage.

FIG. 4 is a schematic, partly sectional illustration showing endoscope 22 in sleeve 26 as the endoscope within the sleeve is retracted from the body passage, in accordance with an embodiment of the present invention. During an endoscopic examination or other procedure, the operator frequently moves the endoscope forward and back in order to return to areas of the body passage that were viewed previously. While the endoscope is moved back (i.e., retracted) through the body passage, sleeve 26 is deflated and thus collapses against endoscope 22, as shown in FIG. 4. Preferably, suction is applied through channel 40 so that the sleeve adheres to the endoscope surface. This adhesion prevents relative motion between the endoscope and the sleeve, so that distal end 30 of endoscope 22 remains in engagement with distal end 28 of the sleeve and does not slide back out of the sleeve.

As endoscope 22 is retracted from the body passage, the endoscope passes backward out of dispenser 24 through entrance port 36 in anchor 34. Because of the snug fit of the anchor around the endoscope, the anchor acts as a capture mechanism for sleeve 26, causing the sleeve to bunch in a capture region 42 within the dispenser. Thus, no part of the sleeve is allowed to exit proximally from the dispenser, and any contamination that has been picked up by the sleeve within the body passage remains contained within the dispenser. As a result, all areas outside the patient's body, including the examination table, the operator's hands and the controls of the endoscope (not shown) are also protected from contamination.

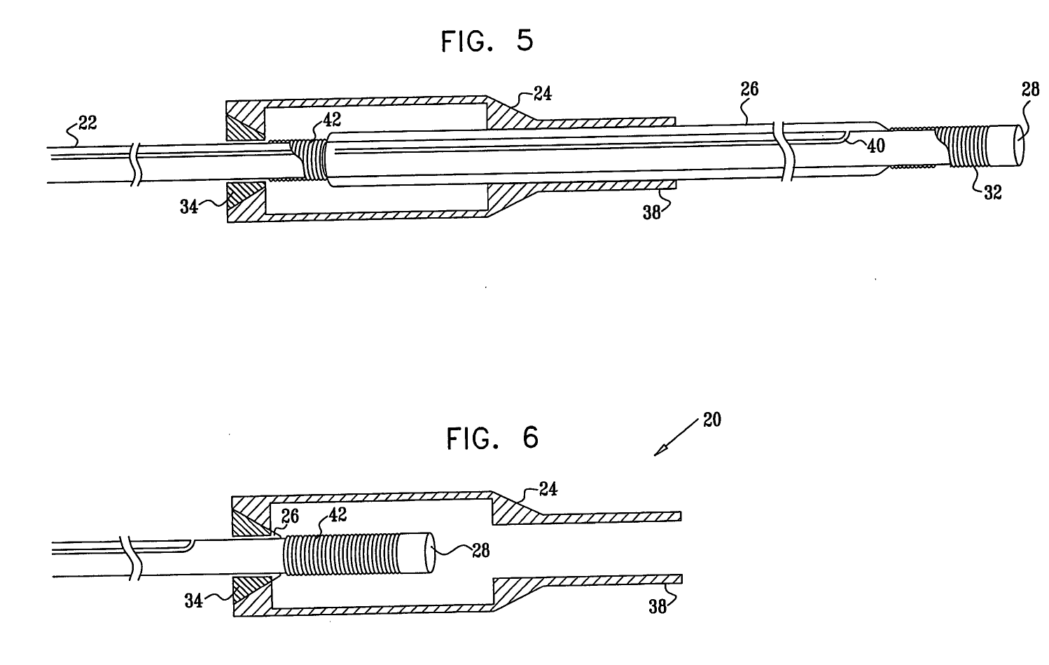

FIG. 5 is a schematic, partly sectional illustration showing endoscope 22 again advancing into the body passage after having been partly retracted, in accordance with an embodiment of the present invention. Sleeve 26 is reinflated through channel 40, causing more of the sleeve to unfold from the distal compaction region 32 as the endoscope advances. At the same time, the inflated sleeve pushes outward against neck 38 of dispenser 24. The friction thus engendered between the sleeve and the distal neck prevents the bunched sleeve in capture region 42 from unfolding. Therefore, the sleeve feeds out only from distal compaction region 32 and remains largely stationary relative to the wall of the body passage as the endoscope advances, in the manner described above. The distal end of neck 38 may be further narrowed at exit port 39 in order to catch sleeve 26 and ensure that the sleeve feeds out evenly from compaction region 32 whenever the endoscope is advanced into the body passage.

FIG. 6 is a schematic, partly sectional illustration showing sleeve assembly 20 after endoscope 22 has been withdrawn completely from the body passage, in accordance with an embodiment of the present invention. At this stage, all of sleeve 26 is bunched in capture region 42 inside dispenser 24. Once the endoscope is pulled back completely out of the sleeve, assembly 20 can simply be disposed of. Since no part of the endoscope, the operator's hands or the examination table has come into any contact with contamination from inside the body passage, the endoscope can be cleaned off quickly and easily, and no disinfection is required.

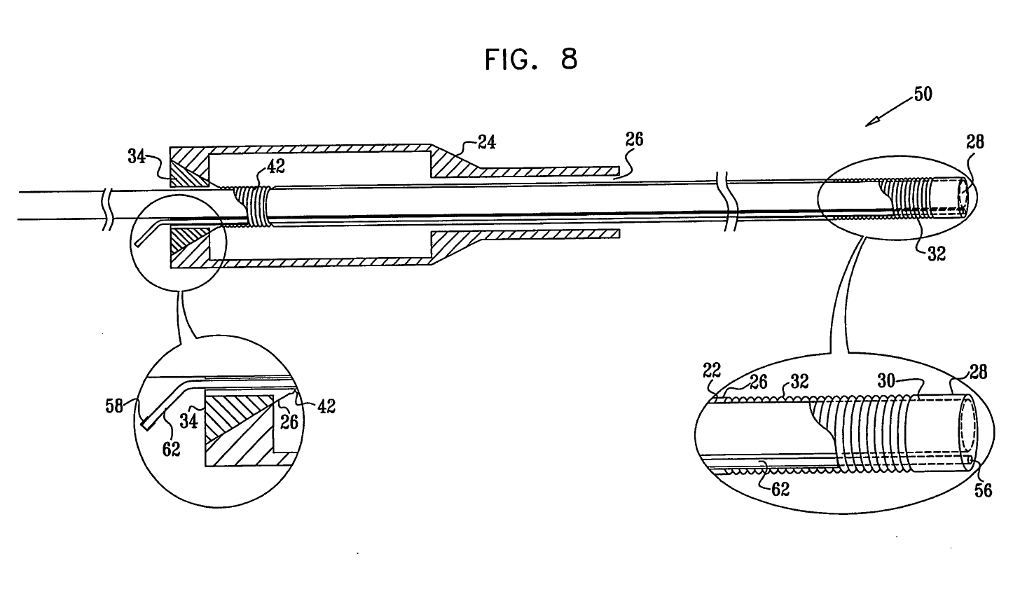

In the embodiment described above, for the sake of simplicity, no working channels are shown. Such working channels may be used, as is known in the art, for suction or irrigation in the area of distal end 30 of the endoscope, as well as for passing endoscopic tools, such as biopsy forceps or snares, to and from the distal region. Sleeve assembly 20 may be modified to accommodate one or more working channels, as described below with reference to FIGS. 7 and 8. Methods for sheathing an endoscope while providing working channels that will not contaminate the endoscope are also described, for example, in the above-mentioned U.S. Pat. No. 4,646,722, as well as in U.S. Pat. No. 4,741,326, whose disclosure is incorporated herein by reference.

FIG. 7 is a schematic, partly sectional illustration of a sleeve assembly 50 for endoscope 22 that includes a working channel 52, in accordance with an embodiment of the present invention. Assembly 50 comprises both an external sleeve 26, which fits over the outside of endoscope 22 as described above, and an internal sleeve 54, which fits inside working channel 52. The distal extremity of internal sleeve 54 communicates with the region distal to endoscope 22 through an opening 56 in distal end 28 of sleeve 26. Before the operator begins to advance the endoscope (covered by sleeve 26) into the body passage, internal sleeve 54 is first threaded through working channel 52 to an outlet 57 from endoscope 22. The proximal extremity of sleeve 54 may then be connected to a source of irrigation or suction, as appropriate, or may be used to pass endoscopic tools through to opening 56 at the distal end. The endoscope, covered by sleeve 26, is then advanced into and retracted from the body passage in the manner described above. Internal sleeve 54 prevents contamination from within the body passage from contacting the interior of working channel 52.

After the endoscopic procedure is completed, endoscope 22 is retracted, and sleeve 26 bunches within dispenser 24 in the manner shown in FIG. 6. Internal sleeve 54 is then pulled out in the distal direction through working channel 52. To prevent contamination of the working channel by the proximal extremity of the internal sleeve as the proximal extremity is pulled through the working channel, a tightly-fitting cap 58 is placed over the proximal extremity of sleeve 54 in order to seal off the sleeve. Alternatively or additionally, the proximal extremity of sleeve 54 may be crimped or heat-sealed in order to prevent any liquids or other debris from leaking out of sleeve 54 into working channel 52.

FIG. 8 is a schematic, partly sectional illustration of a sleeve assembly 60 for endoscope 22, in accordance with another embodiment of the present invention. In this embodiment, sleeve 26 contains a disposable working channel 62, which runs alongside endoscope 22 within the sleeve, rather than passing through the endoscope. The proximal extremity of channel 62 passes through anchor 34 and can be connected to a source of irrigation or suction or used to pass tools through to opening 56 at distal end 28 of sleeve. At the conclusion of the endoscopic procedure, the proximal extremity of channel 62 may be sealed off, as described above, to prevent contamination of endoscope 22 when the endoscope is removed from sleeve assembly 60.

In a further embodiment of the present invention, not shown in the figures, a sleeve assembly may provide both an internal sleeve, such as sleeve 54, which fits inside a working channel within the endoscope, and an external working channel, such as channel 62.

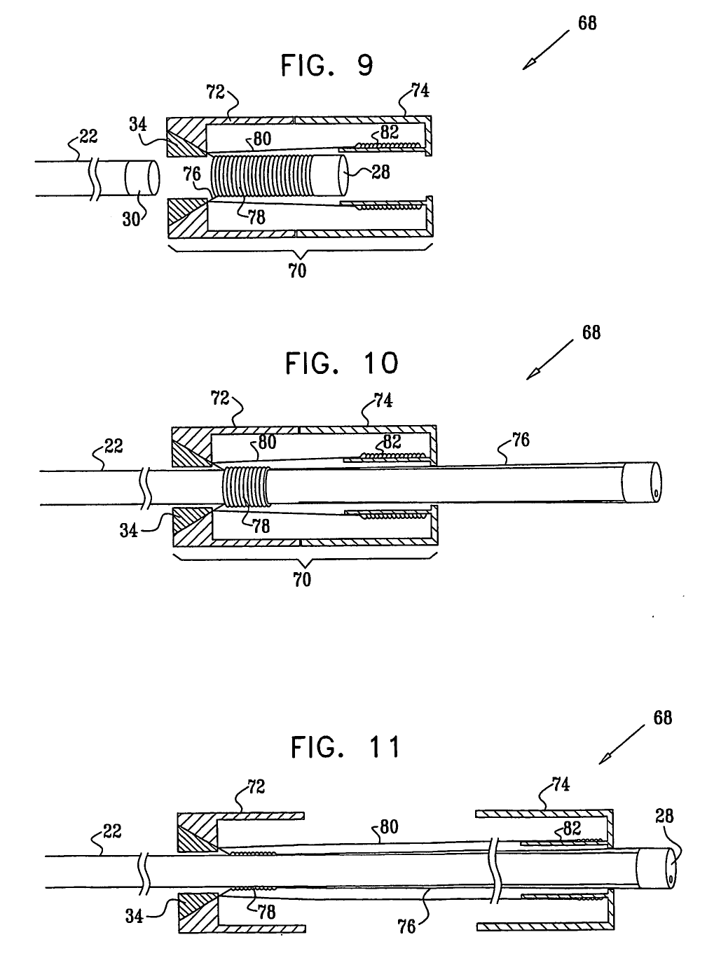

FIG. 9 is a schematic, partly sectional illustration of a sleeve assembly 68 for endoscope 22, in accordance with an alternative embodiment of the present invention. Assembly 68 comprises a dispenser 70, which is divided into a proximal section 72 and a distal section 74. An inner sleeve 76 is held by anchor 34 in proximal section 72. Inner sleeve 76 is initially bunched in an inner compaction region 78, typically using an accordion fold as described above. The inner sleeve is surrounded by an outer sleeve 80, which is fixed to distal section 74 and is bunched in an outer compaction region 82. Before endoscope 22 is advanced into the body passage of interest, distal end 30 of endoscope is first inserted into inner sleeve 76 so that it engages distal end 28 of the inner sleeve, as described above.

FIG. 10 is a schematic, partly sectional illustration showing endoscope 22 inside sleeve 76 as the sleeved endoscope advances into the body passage, in accordance with an embodiment of the present invention. In this embodiment, assembly 68 is designed so that compaction region 78 remains behind inside dispenser 70, and sleeve 76 unfolds in the distal direction as endoscope 22 advances. Distal section 74 of the dispenser may have a narrowed neck 84, so as to ensure that sleeve 76 opens evenly out of the dispenser. Alternatively, the sleeve and dispenser may be configured so that the compaction region advances with the distal end of the endoscope, and the sleeve opens out in the proximal direction as in the preceding embodiments.

FIG. 11 is a schematic, partly section illustration showing deployment of outer sleeve 80 as endoscope 22 is retracted from the body passage, in accordance with an embodiment of the present invention. Before retracting the endoscope, the operator detaches proximal section 72 from distal section 74 of dispenser 70. While retracting the endoscope, the operator pulls proximal section 72 back, away from the body opening, together with the endoscope. At the same time, the operator pushes distal section 74 forward, against the patient's body. As the proximal section moves back, away from the distal section, outer sleeve 80 is deployed in the proximal direction out of compaction region 82, thus covering and containing inner sleeve 76. Thus, any contamination on the outer surface of sleeve 76 is contained within the outer sleeve, and does not contact the endoscope or any other objects or areas outside the patient's body. After endoscope 22 has been removed entirely from sleeve 76, assembly 68 is discarded. This arrangement provides added protection against spread of contamination from inner sleeve 76 to the operator's hands or other areas outside the patient's body.

Embodiments of the present invention are particularly useful in the field of colonoscopy, as noted earlier, but the sleeve assemblies described above may similarly be adapted for use in other applications of flexible endoscopes. Furthermore, the principles of the present invention may be applied, mutatis mutandis, to sleeve other invasive medical tools, such as catheters and rigid endoscopes, as well as probes of other types that come into contact with hazardous materials. It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. Apparatus for sheathing an endoscope, comprising:
a dispenser, having entry and exit ports defining a transit passage through which the endoscope may pass; and
a flexible sleeve, at least a portion of which is bunched in a vicinity of the dispenser, the sleeve comprising a distal end, which is closed, and a proximal end, which is open and fixed to the dispenser so that as the endoscope is advanced in a distal direction through the transit passage, the endoscope enters into the sleeve through the proximal end and engages the distal end of the sleeve, thus causing the bunched portion of the sleeve to be extended so as to cover a distal part of the endoscope that protrudes through the exit port.

2. The apparatus according to claim 1, wherein the dispenser is adapted to be placed so that the exit port is adjacent to a body opening of a patient, whereby the distal part of the endoscope that is covered by the sleeve extends through the body opening into a body passage of the patient.

3. The apparatus according to claim 1, wherein the bunched portion of the sleeve is adjacent to the distal end of the sleeve, and the sleeve is adapted to extend away from the bunched portion in a proximal direction so as to cover the distal part of the endoscope as the endoscope is advanced.

4. The apparatus according to claim 3, wherein the dispenser is adapted to capture the sleeve as the endoscope is retracted through the transit passage in the proximal direction, so that the sleeve is removed from a proximal part of the endoscope that has been retracted through the entry port, and the removed sleeve is gathered in the dispenser.

5. The apparatus according to claim 4, wherein the sleeve is adapted so that after the endoscope has been retracted, whereby the sleeve is removed from the proximal part of the endoscope, a further part of the sleeve extends away from the bunched portion in the proximal direction so as to cover the distal part of the endoscope as the endoscope is again advanced through the transit passage.

6. The apparatus according to claim 1, wherein the dispenser is adapted to capture the sleeve as the endoscope is retracted through the transit passage in a proximal direction, so that the sleeve is removed from a proximal part of the endoscope that has been retracted through the entry port, and the removed sleeve is gathered in the dispenser.

7. The apparatus according to claim 6, wherein the dispenser is adapted to gather substantially all of the sleeve, so that after the distal part of the endoscope has been retracted through the entry port, substantially all of the sleeve is contained within the dispenser.

8. The apparatus according to claim 1, and comprising an external sleeve, fixed to the dispenser, which is adapted to be extended from the dispenser when the endoscope is retracted through the transit passage, so that the external sleeve covers the flexible sleeve that was extended to cover the distal part of the endoscope.

9. The apparatus according to claim 8, wherein the dispenser comprises a proximal section, which defines the entry port and to which the flexible sleeve is fixed, and a distal section, which defines the exit port and to which the external sleeve is fixed, and wherein the distal section is adapted to be moved away from the proximal section of the dispenser so as to extend the external sleeve over the flexible sleeve when the endoscope is retracted through the transit passage.

10. The apparatus according to any of claims 1-9, wherein the sleeve is adapted to be inflated while the endoscope is advanced through the transit passage.

11. The apparatus according to claim 10, wherein the sleeve is adapted to be deflated while the endoscope is retracted proximally through the transit passage.

12. The apparatus according to claim 11, wherein the dispenser comprises a channel, communicating with the sleeve, for inflating the sleeve while the endoscope is advanced and for applying suction to the sleeve while the endoscope is retracted.

13. The apparatus according to claim 10, wherein the entry port is adapted to fit snugly around the endoscope so as to prevent escape of pressure through the entry port when the sleeve is inflated.

14. The apparatus according to any of claims 1-9, and comprising a working channel extending through the sleeve alongside the endoscope, the working channel comprising distal and proximal extremities, wherein the distal extremity is fixed to the distal end of the sleeve, and the proximal extremity protrudes from the dispenser.

15. The apparatus according to claim 14, and comprising a sealing element, which is adapted to seal the proximal extremity of the working channel while the endoscope is removed from the dispenser.

16. The apparatus according to any of claims 1-9, wherein the endoscope includes a working channel having distal and proximal outlets, and wherein the apparatus comprises an internal sleeve, which is adapted to be inserted through the working channel, the internal sleeve comprising distal and proximal extremities, wherein the distal extremity is fixed to the distal end of the sleeve, and the proximal extremity protrudes from the proximal outlet of the working channel.

17. The apparatus according to claim 16, and comprising a sealing element, which is adapted to seal the proximal extremity of the internal sleeve while the endoscope is removed from the dispenser.

18. Apparatus for endoscopy, comprising:
an endoscope, which is adapted to be inserted into a body passage of a patient; and
a sleeve assembly, which comprises:
a dispenser, having entry and exit ports defining a transit passage through which the endoscope may pass; and
a flexible sleeve, at least a portion of which is bunched in a vicinity of the dispenser, the sleeve comprising a distal end, which is closed, and a proximal end, which is open and fixed to the dispenser so that as the endoscope is advanced in a distal direction through the transit passage, the endoscope enters into the sleeve through the proximal end and engages the distal end of the sleeve, thus causing the bunched portion of the sleeve to be extended so as to cover a distal part of the endoscope that protrudes through the exit port.

19. The apparatus according to claim 18, wherein the endoscope is adapted to be inserted into the body passage through a body opening of the patient, and wherein the dispenser is adapted to be placed with the exit port adjacent to the body opening, so that the distal part of the endoscope is covered by the sleeve as the endoscope passes through the body opening into the body passage.

20. The apparatus according to claim 18, wherein the bunched portion of the sleeve is adjacent to the distal end of the sleeve, and the sleeve is adapted to extend away from the bunched portion in a proximal direction so as to cover the distal part of the endoscope as the endoscope is advanced.

21. The apparatus according to claim 20, wherein the dispenser is adapted to capture the sleeve as the endoscope is retracted through the transit passage in the proximal direction, so that the sleeve is removed from a proximal part of the endoscope that has been retracted through the entry port and is gathered in the dispenser.

22. The apparatus according to claim 21, wherein the sleeve is adapted so that after the endoscope has been retracted, whereby the sleeve is removed from the proximal part of the endoscope, a further part of the sleeve extends away from the bunched portion in the proximal direction so as to cover the distal part of the endoscope as the endoscope is again advanced in the distal direction through the transit passage.

23. The apparatus according to claim 18, wherein the dispenser is adapted to capture the sleeve as the endoscope is retracted through the transit passage in a proximal direction, so that the sleeve is removed from a proximal part of the endoscope that has been retracted through the entry port and is gathered in the dispenser.

24. The apparatus according to claim 23, wherein the dispenser is adapted to gather substantially all of the sleeve, so that after the distal part of the endoscope has been retracted through the entry port, substantially all of the sleeve is contained within the dispenser.

25. The apparatus according to claim 18, wherein the sleeve assembly comprises an external sleeve, fixed to the dispenser, which is adapted to be extended from the dispenser when the endoscope is retracted through the transit passage, so that the external sleeve covers the flexible sleeve that was extended to cover the distal part of the endoscope.

26. The apparatus according to claim 25, wherein the dispenser comprises a proximal section, which defines the entry port and to which the flexible sleeve is fixed, and a distal section, which defines the exit port and to which the external sleeve is fixed, and wherein the distal section is adapted to be moved away from the proximal section of the dispenser so as to extend the external sleeve over the flexible sleeve when the endoscope is retracted through the transit passage.

27. The apparatus according to any of claims 18-27, and comprising a channel, communicating with the sleeve, for inflating the sleeve while the endoscope is advanced in the distal direction through the transit passage.

28. The apparatus according to claim 27, wherein the channel is further adapted for applying suction to the sleeve while the endoscope is retracted proximally through the transit passage.

29. The apparatus according to claim 27, wherein the entry port is adapted to fit snugly around the endoscope so as to prevent escape of pressure through the entry port when the sleeve is inflated.

30. The apparatus according to claim 27, wherein the channel is contained within the endoscope.

31. The apparatus according to claim 27, wherein the channel is contained within the dispenser, alongside the endoscope.

32. The apparatus according to any of claims 18-27, wherein the sleeve assembly comprises a working channel extending through the sleeve alongside the endoscope, the working channel comprising distal and proximal extremities, wherein the distal extremity is fixed to the distal end of the sleeve, and the proximal extremity protrudes from the dispenser.

33. The apparatus according to claim 32, and comprising a sealing element, which is adapted to seal the proximal extremity of the working channel while the endoscope is removed from the dispenser.

34. The apparatus according to any of claims 18-27, wherein the endoscope comprises a working channel having distal and proximal outlets, and wherein the sleeve assembly comprises an internal sleeve, which is adapted to be inserted through the working channel, the internal sleeve comprising distal and proximal extremities, wherein the distal extremity is fixed to the distal end of the sleeve, and the proximal extremity protrudes from the proximal outlet of the working channel.

35. The apparatus according to claim 34, and comprising a sealing element, which is adapted to seal the proximal extremity of the internal sleeve while the endoscope is removed from the dispenser.

36. A method for protecting an endoscope from contamination, comprising:
providing a flexible sleeve comprising a distal end, which is closed, and a proximal end, which is open, wherein at least a portion of the sleeve is bunched in a compaction region;
inserting a distal part of the endoscope into the proximal end and through the bunched portion of the sleeve so as to engage the distal end; and
advancing the endoscope through a body opening of a patient into a body passage while extending the bunched portion of the sleeve so as to cover the distal part of the endoscope that extends through the body opening into the body passage.

37. The method according to claim 36, wherein advancing the endoscope comprises placing the compaction region adjacent to the body opening, so that advancing the endoscope into the body passage of the patient causes the sleeve to unfold from the compaction region in order to cover the distal part of the endoscope.

38. The method according to claim 37, wherein the bunched portion of the sleeve is adjacent to the distal end of the sleeve, and wherein advancing the endoscope comprises causing the sleeve to extend away from the bunched portion in a proximal direction so as to cover the distal part of the endoscope as the endoscope is advanced.

39. The method according to claim 36, and comprising retracting the endoscope in a proximal direction, and capturing the sleeve while retracting the endoscope so that the sleeve is removed from a proximal part of the endoscope and is gathered in a location adjacent to the body opening.

40. The method according to claim 39, wherein capturing the sleeve comprises gathering substantially all of the sleeve in a receptacle, so that after the distal part of the endoscope has been retracted from the body passage, substantially all of the sleeve is contained within the receptacle.

41. The method according to claim 36, and comprising retracting the endoscope in a proximal direction, while extending an external sleeve to cover the flexible sleeve as the endoscope is withdrawn from the body passage.

42. The method according to any of claims 36-41, wherein advancing the endoscope comprises inflating the sleeve while the endoscope is advanced in into the body passage.

43. The method according to claim 42, and comprising retracting the endoscope from the body passage, and applying suction to the sleeve while the endoscope is retracted.

44. The method according to any of claims 3641, wherein the sleeve comprises a working channel extending through the sleeve alongside the endoscope, the working channel comprising distal and proximal extremities, wherein the distal extremity is fixed to the distal end of the sleeve, and the proximal extremity protrudes from the body opening.

45. The method according to claim 44, and comprising sealing the proximal extremity of the working channel before removing the sleeve from the endoscope.

46. The method according to any of claims 36-41, wherein the endoscope comprises a working channel having distal and proximal outlets, and wherein the sleeve comprises an outer sleeve, for covering the distal part of the endoscope, and an internal sleeve comprising distal and proximal extremities, wherein the distal extremity is fixed to the distal end of the outer sleeve, and wherein the method comprises inserting the internal sleeve through the working channel so that the proximal extremity protrudes from the proximal outlet of the working channel.

47. The method according to claim 46, and comprising sealing the proximal extremity of the internal sleeve before removing the internal sleeve from the working channel.
